SEQUENCE LISTING (4 sequences)

SEQ ID NO: 1 - DNA, 51 bp, Homo sapiens
tttatcacactccatgatcccagctkctaaaatccacactgagctctgctt

SEQ ID NO: 2 - DNA, 808 bp, Homo sapiens
gagatggcagctggcttggcaaggggacagcacctttgtcaccacattatgtccctgtaccctacatgctgcgcctatacccaggaccgatggtaactgaggcggaggggaaaggagggcctgagatggcaagtctgtcctcctcggtggttcctgtgtccttcatttccactctgcgagagtctgtgctggaccctggagttggtggagaaggagccagtgacaagcagaggagcaaactgtctttatcacactccatgatcccagctgctaaaatccacactgagctctgcttaccagccttcttctctcctgctggaacccagaggaggttccagcagcctcagcaccacctgacactgtctatcatccacactgcagcaaggtgattctgccaaaacatatctccttaaaagccaactggagcttctcatcagcatcaatgtgaagccaaaaatccttaggaggacagagggagtccctcacaacctagactggtccccttccctccagctgcctcaactgtccacaggactctcttcccacctgcggccacactgtgcaacctggaatttccccacctgggcggactcatcacgtcatcaccaattggatgcatcttctgctctgtgcagctggtgaaatctttctcaacccttgagatgcagcccaatcttctcctaacatctggattcctctctgtcactgcattccctcctgtcatcctgcctttgttttcttgccctcctttctctcccgggtgataggcattaactaaaattaaataaaaattcagatcatccttgca

SEQ ID NO: 3 - DNA, 23 bp, Homo sapiens
ggtttcttcttgaaaatcacagg

SEQ ID NO: 4 - DNA, 22 bp, Homo sapiens
ccattggtaaaacaaggtgaca

(19) United States
(12) Patent Application Publication
(10) Pub. No.: US 2011/0183340 A1
(43) Pub. Date: Jul. 28, 2011

(54) Phenotype-Genotype Relationship in Age-Related Macular Degeneration

(75) Inventors: R. Keith Shuler, JR., Pinehurst, NC (US); Eric A. Postel, Durham, NC (US); Jennifer Caldwell, Durham, NC (US); Silke Schmidt, Durham, NC (US); Michael Hauser, Durham, NC (US)

(73) Assignee: DUKE UNIVERSITY, Durham, NC (US)

(21) Appl. No.: 13/036,780

(22) Filed: Feb. 28, 2011

Related U.S. Application Data
(63) Continuation of application No. 12/348,167, filed on Jan. 2, 2009, now abandoned.
(60) Provisional application No. 61/018,997, filed on Jan. 4, 2008.

(51) Int. Cl. C12Q 1/68; G01N 33/53

(52) U.S. Cl. 435/6.12; 435/6.1; 435/7.1; 435/7.92

(57) ABSTRACT

Age-Related Macular Degeneration (AMD) cases possessing the LOC387715 (rs 10490924) variant have a higher risk of neovascular AMD. Individuals with AMD who are homozygous for both variants might be at greater risk for earlier onset of neovascular AMD. Determining the presence of this variant indicates which path the disease may take and which nutritional, supplement, or medicaments are appropriate.

This invention was made using funds from U.S. government grant no. U10EY1218-05 from the National Institutes of Health (NIH)/National Eye Institute. Therefore the U.S. government retains certain rights in the invention.

### TECHNICAL FIELD OF THE INVENTION

This invention is related to the area of genetic testing, drug discovery, and Age-Related Macular Degeneration. In particular, it relates to genetic variants which increase the risk of Age-Related Macular Degeneration.

### BACKGROUND OF THE INVENTION

Age-related macular degeneration (AMD) is the leading cause of irreversible central vision loss in older Americans.¹ The clinical characteristics of AMD are generally divided into nonneovascular and neovascular forms. Previously described phenotypic characteristics associated with neovascular AMD include white race, increasing age, increased body mass index, hypertension, hyperopia, intraocular pressure, lens opacity, and large drusen.²⁻³

Recent articles have shown that a common polymorphism of the complement factor H gene (CFH) (T1277C at rs1061170, or Y402H) is associated with macular soft drusen⁴ as well as an increased risk of advanced AMD, including geographic atrophy and neovascular AMD.⁴⁻⁸ One recent article⁸ suggested that the CFH variant increases the risk for geographic atrophy in particular. The CFH Y402H polymorphism also is associated with peripheral reticular pigmentary changes.⁹

A second putative AMD susceptibility gene, LOC387715 (T allele at rs10490924, or A69S), has recently been identified.¹⁰ Biological characterization of this gene is limited; however, smokers with this LOC387715 variant have a substantially greater risk for advanced AMD, especially the neovascular form, compared with nonsmokers with this variant.¹¹

Clarifying the phenotype-genotype relationships in AMD might provide clues to the involved molecular mechanisms and may guide treatment recommendations for specific subtypes of AMD. Here we compare the phenotypes associated with two recently described AMD risk genes: LOC387715 and CFH variants.

There is a continuing need in the art to identify individual genes that are useful for the stratification, prediction of risk, and assignment of appropriate nutritional or medicament regimens.

### SUMMARY OF THE INVENTION

According to one embodiment of the invention a method is provided for assessing risk of neovascular AMD in a patient. The presence of a T allele at rs10490924 in LOC387715 in the patient's genome is determined. The patient is identified as being at higher risk of neovascular AMD than geographical atrophy or of being at higher risk of neovascular AMD than a patient without the T allele.

According to another embodiment of the invention a method is provided for assessing risk of neovascular AMD in a patient. The presence of two T alleles at rs10490924 in LOC387715 in the patient's genome is determined The patient is identified as being at higher risk of neovascular AMD than geographical atrophy or of being at higher risk of neovascular AMD than a patient without the T allele and as having an earlier predicted onset of neovascular AMD than a patient with one T allele at rs10490924.

Yet another embodiment of the invention provides a method to determine an appropriate regimen for prescribing to a patient for slowing progression or delaying onset of neovascular AMD. A patient is tested to determine the presence of two T alleles at rs10490924 in LOC387715. A patient with two T alleles at rs10490924 is identified as having an earlier predicted onset of neovascular AMD than a patient with one or no T allele at rs10490924. A medicament, supplement, or diet is prescribed for the patient with two T alleles at rs 10490924 to delay onset or slow progression of neovascular AMD.

These and other embodiments of the invention provide the art with additional tools for recognizing and stratifying patients for risk and prevention of neovascular AMD.

### BRIEF DESCRIPTION OF THE DRAWINGS

FIG. 1 provides a description of the study population by genotypic group, detailed infra.

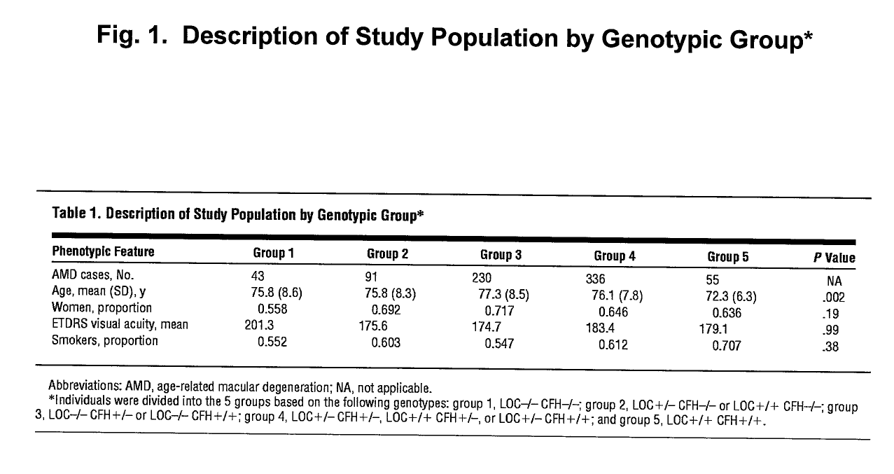

FIG. 2 provides a comparison of proportion of age-related macular degeneration cases in each phenotypic grade by genotypic group.

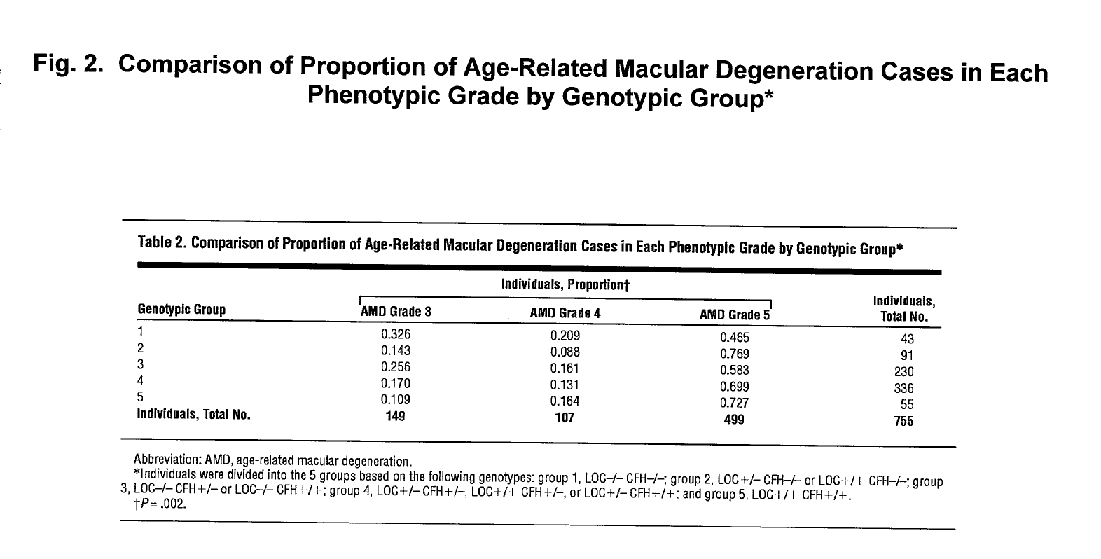

FIG. 3 provides the proportion of individuals with a specific phenotypic feature by genotypic group.

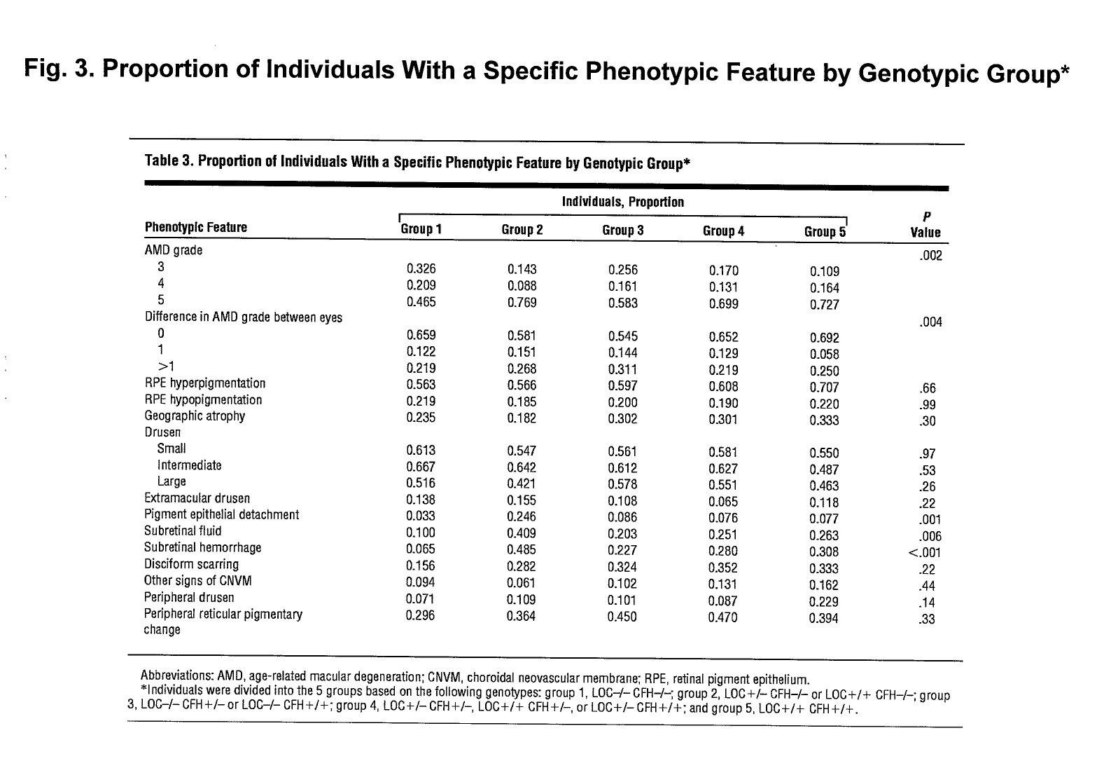

### DETAILED DESCRIPTION OF THE INVENTION

The inventors set out to compare phenotypes of two age-related macular degeneration (AMD) susceptibility genes: LOC387715 (chromosome 10q26) and complement factor H (CFH; chromosome 1q32). Phenotypes of 755 AMD cases were characterized. The number of LOC387715 (T allele at rs 10490924, or A69S) and CFH (T1277C at rs1061170, or Y402H) risk alleles were determined in each case. Individuals were divided into five groups by genotype:

| | |
|---|---|
| group 1 | LOC−/− CFH−/− |
| group 2 | LOC+/− CFH−/− or LOC+/+ CFH−/− |
| group 3 | LOC−/− CFH+/− or LOC−/− CFH+/+ |
| group 4 | LOC+/− CFH+/−, LOC+/+ CFH+/−, or LOC+/− CFH+/+ |
| group 5 | LOC+/+ CFH+/+ |

The inventors observed that signs of neovascular AMD including grade (P=0.002), pigment epithelial detachment (P=0.001), and subretinal hemorrhage (P<0.001) demonstrated significant association with groups 2, 4, and 5 versus groups 1 and 3. Group 5 had a significantly younger mean age (72.3 years) compared with other groups (P=0.002). The AMD cases possessing the LOC387715 (rs10490924) variant have a higher risk of neovascular AMD. Individuals with AMD who are homozygous for both variants are at greater risk for earlier onset of neovascular AMD.

AMD is graded using a number of clinical observations. These phenotypic distinctions are: grade 1 is defined as no drusen or small (<63 um) nonextensive drusen without RPE abnormalities; grade 2 is defined as extensive small drusen or non-extensive intermediate drusen (>63 um, <125 um) and/or retinal pigment epithelium hyper or hypopigmentation, but not geographic atrophy; grade 3 was defined as extensive intermediated drusen or any large, soft drusen (>125 um), including drusenoid retinal pigment epithelial detachment; grade 4 is defined as geographic atrophy (area of RPE atrophy with sharp margins, usually visible choroidal vessels, at least 175 um diameter); and grade 5 is exudative AMD, including nondrusenoid pigment epithelium detachment, CNVM, subretinal hemorrhage or fibrosis, or a photocoagulation scar consistent with treatment of AMD.

The two variants (the Y402H polymorphism of CFH and the T allele of LOC387715 at rs10490924) are known in the art and can be determined by any means known in the art. The variant can be determined by analysis of genomic DNA or by analysis of mRNA or by analysis of protein. Any convenient and expedient technique can be used, including but not limited to ELISA, immunoblot, immunoprecipitation, PCR, real-time PCR, rolling circle amplification, nucleotide or amino acid sequence determination, primer mass extension and matrix-assisted laser desorption ionization-time-of-flight mass spectrometry analysis. The recording of the result may be done by a machine or computer. DNA can be analyzed to determine its sequence using a synthetic technique in which DNA is synthesized using an analyte template. Alternatively, DNA can be analyzed using a degradative technique in which an analyte is chemically, mechanically, or enzymatically cleaved and the degradation products are analyzed. Patient samples which may be used include any tissue or body fluid which provides either DNA or protein in a suitable condition for analysis.

As is known in the medical arts and sciences, a single diagnostic or prognostic parameter may or may not be relied upon in isolation. A number of different parameters may be considered in combination, including but not limited to patient age, general health status, sex, lifelong health habits, medication history, and physical or clinical findings. The latter may include, macular or extramacular drusen, retinal pigment epithelial changes, subretinal fluid, subretinal hemorrhage, disciform scarring, subretinal exudate, peripheral drusen, and peripheral reticular pigmentary change.

Smoking behavior can be scored in a number of ways. One way is as ever/never. Another option is to determine a quantitative measure, such as pack-years of smoking over a life time. Another possible criterion is whether a patient is a current smoker; current smokers may be scored as individuals who smoke at least once per week. Past smokers are another category that can be scored.

The Y402H polymorphism of CFH is encoded by the T1277C polymorphism. The T allele of LOC387715 is the variant of rs10490924 that has a T at nucleotide 26 as shown in SEQ ID NO: 1 (tttatcacac tccatgatcc cagctkctaa aatccacact gagctctgct t). The entire non-variant nucleotide sequence of LOC387715 is:

| (SEQ ID NO: 2) | |
|---|---|
| gagatggcag ctggcttggc aaggggacag cacctttgtc accacattat gtccctgtac | 60 |
| cctacatgct gcgcctatac ccaggaccga tggtaactga ggcggagggg aaaggagggc | 120 |
| ctgagatggc aagtctgtcc tcctcggtgg ttcctgtgtc cttcatttcc actctgcgag | 180 |
| agtctgtgct ggaccctgga gttggtggag aaggagccag tgacaagcag aggagcaaac | 240 |
| tgtctttatc acactccatg atcccagctg ctaaaatcca cactgagctc tgcttaccag | 300 |
| ccttcttctc tcctgctgga acccagagga ggttccagca gcctcagcac cacctgacac | 360 |
| tgtctatcat ccacactgca gcaaggtgat tctgccaaaa catatctcct taaaagccaa | 420 |
| ctggagcttc tcatcagcat caatgtgaag ccaaaaatcc ttaggaggac agagggagtc | 480 |
| cctcacaacc tagactggtc cccttccctc cagctgcctc aactgtccac aggactctct | 540 |
| tcccacctgc ggccacactg tgcaacctgg aatttcccca cctgggcgga ctcatcacgt | 600 |
| catcaccaat tggatgcatc ttctgctctg tgcagctggt gaaatctttc tcaacccttg | 660 |
| agatgcagcc caatcttctc ctaacatctg gattcctctc tgtcactgca ttccctcctg | 720 |
| tcatcctgcc tttgttttct tgccctcctt tctctcccgg gtgataggca ttaactaaaa | 780 |
| ttaaataaaa attcagatca tccttgca | 808 |

When a risk of neovascular AMD is identified or an early onset of neovascular AMD is identified, patients can be grouped appropriately, i.e., stratified so that appropriate conclusions can be drawn in clinical studies. Additionally, appropriate modifications to lifestyle can be recommended, including, but not limited to diet, supplementation of vitamins and minerals, for example, smoking cessation, drugs, and obesity reduction or control. Supplementation of diet, including but not limited to vitamins C, E, beta carotene, zinc, and/or lutein/zeaxanthin may be recommended. Diets high in these factors may be used as a source of the helpful factors. One particular combination supplement includes: 500 milligrams of vitamin C, 400 milligrams of vitamin E, 15 milligrams of beta-carotene, 80 milligrams of zinc as zinc oxide, two milligrams of copper as cupric oxide. Drugs which may delay onset or reduce progression of disease when it occurs include anti-inflammatory medicaments. Many are known in the art and can be used. Positive dietary recommendations include carrots, corn, kiwi, pumpkin, yellow squash, zucchini squash, red grapes, green peas, cucumber, butternut squash, green bell pepper, celery, cantaloupe, sweet potatoes, dried apricots, tomato and tomato products, dark green leafy vegetables, spinach, kale, turnips, and collard greens.

Identifying a patient with a diagnosis or prognosis typically involves an act of communicating a result or conclusion based on data interpretation. The form of communication may be in writing, oral, or electronic. The communication may be to the patient or patient's family member or caregiver; to a medical record; to a doctor; to a pharmacist; to a nurse; to an insurer; or to a health maintenance organization.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### Example 1

### Methods

Patients were identified in the clinic populations of the Duke University Eye Center, Durham, N.C., and the Department of Ophthalmology, Vanderbilt University, Nashville, Tenn., or from referrals to the study centers by local ophthalmologists. Information was collected and protected in compliance with the Health Insurance Portability and Accountability Act of 1996 regulations, institutional review board approval was obtained, and all of the patients provided informed consent.

The clinical criteria, grades, and grading methods used to define AMD have previously been described.¹² Age-related findings including drusen, retinal pigment epithelial (RPE) changes, neovascularization, and geographic atrophy were used to diagnose AMD in individuals aged 55 years or older.

Data collection for each individual was performed using a standardized protocol. Complete ocular, medical, and family ocular histories were obtained. Most study participants completed these questionnaires in person with the clinical study coordinator (J.C.). Age and sex were recorded. Height, weight, and blood pressure were measured during the clinical encounter. Separate questionnaires were used to obtain lifelong health habits such as smoking, sunlight exposure, and dietary supplementation as well as current dietary practices. Patients completed these questionnaires typically at home or much less frequently by telephone. The measurement of the proportion of smokers in each group was constructed as a binary “ever or never” variable based on a participant's response to the question, “Have you smoked at least 100 cigarettes in your lifetime?”¹¹ Additional smoking history information, including regular cigarette smoking, was assessed as previously described.¹¹ Each participant received a complete ophthalmic evaluation that included slitlamp examination, biomicroscopy with a handheld 90-diopter (D) lens or fundus contact lens, and (20-D) indirect ophthalmoscopy of the peripheral retina.

The study protocol included a minimum of 3 standard fields of 35-mm color fundus photographs as well as stereo photographs of the disc and macula. Two of us (A.A. and E.A.P.) used the previously described modified Age-Related Eye Disease Study grading system to grade macular findings.¹³⁻¹⁵ Each eye of every individual received a grade. The overall grade for a participant was based on the more severely affected eye; if multiple features appeared within an eye, the more severe grade was applied.

Photographic evaluation was performed using standardized illumination (6000 K) and stereoscopic magnification. Lenses were graded using the Lens Opacities Classification System III standards whenever possible.¹⁶ Detailed information was recorded from clinical and photographic examination regarding extramacular and peripheral (anterior to the equator) drusen, peripheral reticular pigmentary change, posterior vitreous detachment, and iris color.

Individuals with disease of grade 3 or higher in at least 1 eye were considered affected. The reliability of grading has previously been examined, and concordance was found in 92% with a κ score of 0.81.¹² The power to detect a difference of 0.5 or more grade units between the 2 groups was greater than 99%.¹²

Phenotypic features investigated included the following: AMD grade, difference in AMD grade between eyes, ETDRS visual acuity, cataract assessment (existence or absence), RPE hyperpigmentation, RPE hypopigmentation, geographic atrophy, macular drusen (existence, type, or extent as defined by grade [small, medium, and large]), extramacular drusen, pigmented epithelial detachment, subretinal fluid, subretinal hemorrhage, disciform scarring, other signs of choroidal neovascular membrane (e.g., subretinal exudate), peripheral drusen, and peripheral reticular pigmentary change.

The rs1061170 single nucleotide polymorphism (Y402H) falls within a genomic duplication and could not easily be genotyped using TaqMan assays (Applied Biosystems, Foster City, Calif.). All of the samples were sequenced using primers GGTTTCTTCTTGAAAATCACAGG (SEQ ID NO: 3) and CCATTGGTAAAACAAGGTGACA (SEQ ID NO: 4) to determine the CFH genotypes.⁵ Genotyping for the LOC387715 rs10490924 variant was performed using the TaqMan allelic discrimination assay (Applied Biosystems).¹¹

The AMD cases were divided into 5 groups depending on genotype. Group 1 lacked the CFH rs1061170 variant and the LOC387715 rs10490924 variant (LOC−/− CFH−/−). Group 2 was heterozygous or homozygous for the LOC387715 rs10490924 risk variant and lacked the CFH rs1061170 variant (LOC+/− CFH−/− or LOC+/+CFH−/−). Group 3 lacked the LOC387715 rs10490924 variant and was heterozygous or homozygous for the CFH rs1061170 variant (LOC−/− CFH+/− or LOC−/−CFH+/+). Group 4 carried at least 1 risk allele at both loci but was not homozygous for both AMD risk polymorphisms (LOC+/− CFH+/−, LOC+/+ CFH+/−, or LOC+/− CFH+/+). Group 5 was homozygous for both AMD risk loci (LOC+/+ CFH+/+).

Groups were independently analyzed for association with all of the phenotypic characteristics investigated as well as age and sex. Statistical analyses were performed using SAS software version 8.2 (SAS Institute, Inc, Cary, N.C.). Conservatively assuming that all of the 16 comparisons are statistically independent, the Bonferroni correction requires an a level of approximately 0.0031 to achieve statistical significance. For categorical variables, phenotypic differences among the 5 genotype-defined groups were compared with a global χ2 test. For continuous variables, analysis of variance was used.

### Example 2

### Results

The data set contained 755 unrelated AMD cases divided into 5 groups by genotype:

group 1, 43 cases; group 2, 91 cases; group 3, 230 cases; group 4, 336 cases; and group 5, 55 cases (Table 1). Table 1 compares the general characteristics of the groups. The proportion of women in the 5 groups did not vary significantly (P=0.19). The ETDRS visual acuities also demonstrated no statistical difference between groups (P=0.99). The proportion of smokers did not differ significantly between groups (P=0.38). Group 5 had a mean±SD age of 72.3±6.3 years, making that group significantly younger than the other 4 groups (P=0.002) (Table 1).

There were significant differences in the proportions of each grade between groups (P=0.002), specifically in the proportion of grade 5 cases between groups 2 and 3 (P=0.002) (Table 2). Two signs of neovascular AMD, pigment epithelial detachment (P=0.001) and subretinal hemorrhage (P<0.001), demonstrated a statistically significant association with groups possessing at least 1 LOC387715 allele (Table 3). In each of these comparisons, there was a measurable difference between groups 2 and 3, with group 2 having a much larger proportion of patients with the respective signs of neovascular AMD (Table 3).

Another sign of neovascular AMD, subretinal fluid (P=0.006), very nearly reached statistical significance (Table 3). Additionally, the difference in AMD grade between eyes (P=0.004) also very nearly reached statistical significance.

There was no significant difference found between groups with respect to the existence or type of cataract (P=0.57), RPE hyperpigmentation (P=0.66), RPE hypopigmentation (P=0.99), geographic atrophy (P=0.30), macular drusen (existence, type, or extent as defined by grade [small drusen {P=0.97}, medium drusen {P=0.53}, or large drusen {P=0.26}]), extramacular drusen (P=0.22), disciform scarring (P=0.22), other signs of choroidal neovascular membrane (P=0.44), peripheral drusen (P=0.14), and peripheral reticular pigmentary change (P=0.33) (Table 3).

### Example 3

### Comments

The CFH (rs1061170) and LOC387715 (rs10490924) variants are the most significant AMD risk genes described to date.^{5-7,10} This analysis suggests that individuals with AMD possessing 1 or more risk alleles at LOC387715 rs10490924 are more likely to develop neovascular AMD compared with those with AMD who lack this variant. Individuals with AMD who are homozygous for both risk variants might be at greater risk for earlier onset of neovascular AMD; however, our data do not prove this.

Several studies,⁴⁻⁸ including our earlier articles, have demonstrated that the C allele at CFH rs 1061170 is associated with an increased risk of both forms of advanced AMD, neovascular and geographic atrophy. Our most recent analysis⁸ suggests that the CFH rs1061170 variant is somewhat more strongly associated with geographic atrophy (odds ratio, 3.2; P<0.001) than neovascular AMD (odds ratio, 2.5; P<0.001), although the confidence intervals overlap. Consistent with our previous study, the current phenotype analysis, which incorporates joint genotypes at CFH and LOC387715, suggests that the T allele at LOC387715 rs10490924 is more strongly associated with neovascular features whereas the C allele at CFH rs 1061170 is more likely to lead to geographic atrophy (grade 4 disease). Prospective studies of the progression in individuals diagnosed with grade 2 or 3 disease at baseline are needed to confirm this hypothesis.

The age at examination was significantly earlier for group 5, and the standard deviation was smaller for this group. Because individuals often are initially evaluated at the onset of symptoms, these data suggest that individuals homozygous for both risk genes may develop symptomatic disease earlier. The relatively smaller variation regarding the age at examination in the AMD cases homozygous for the CFH rs1061170 and LOC387715 rs10490924 variants may suggest a more severe phenotype, not in terms of disease grade but rather a more consistent and earlier onset of disease. However, smoking history cannot be excluded as a factor contributing to this group's earlier age at onset as discussed later. It is possible that being homozygous for both risk variants combined with cigarette smoking creates the conditions for the “perfect storm.”

The joint effect of two AMD risk genes does not account for all of the signs of AMD. Rather, multiple gene interactions as well as dietary and environmental factors in the setting of the aging process all contribute to the phenotype. Therefore, it is not surprising that only some of the signs of neovascular AMD investigated were significantly associated with the LOC387715 rs10490924 variant. Data collected for each individual represent a single point in time; therefore, an individual with neovascular AMD may demonstrate different signs at different times or never develop certain signs of neovascular AMD. Because these data for each patient represent a point in time, we did not emphasize the near significance of the difference in grade between eyes within a participant. There is a continuum of disease in AMD that usually occurs at different rates in each eye. Patients often are initially evaluated when vision decreases in one eye, and symptoms in the second eye follow sometime later. Our study was not longitudinal; therefore, we could not determine whether this difference reached statistical significance or was lost. It would be interesting to investigate whether the difference in grade between eyes reached statistical significance and whether it could be related to the institution of any therapy.

Our previous article¹¹ suggested that smokers with the LOC387715 rs10490924 variant have a substantially greater risk for advanced AMD, especially the neovascular form, compared with nonsmokers with this allele. In the current study, the proportion of smokers did not differ significantly between groups. Although this study did not aim to evaluate the risk of developing AMD, the relatively similar proportions of smokers across genotype groups suggests that a higher proportion of smokers alone did not account for the increased prevalence of neovascular AMD features in the groups possessing the LOC387715 rs10490924 variant.

Within the data set, pack-years of cigarette smoking were significantly different only between groups 3 and 5 (mean pack-years, 32.8 vs 48.2, respectively; P=0.04; overall group difference, P=0.27). This illustrates that one needs to be careful in concluding that the earlier appearance of neovascular AMD in group 5 is solely due to genetic factors. Instead, this observation is likely due to the combination of heavy cigarette smoking and the double homozygous state of both risk variants.

Age-related macular degeneration is a multifactorial disease, and this analysis does not aim to model the joint effect of smoking, CFH, and LOC387715 on the risk of developing AMD, which was done in our previously reported case-control study.11 Rather, our goal was to more carefully characterize the phenotype-genotype relationship of individuals with AMD who possess various combinations of risk alleles at these two genes.

Because these two sequence variants were already demonstrated to confer a greater risk of severe AMD, this study included only individuals with AMD. Our aim was to analyze the phenotypic characteristics of those individuals with AMD who possess one or both of these two risk alleles. However, it is important to realize that not every individual who possesses one of these risk variants is diagnosed with AMD; therefore, it is also relevant to determine the prevalence of these risk variants in the general population or in individuals without AMD. There were 200 of 208 individuals without signs of AMD (grade 1 and grade 2 combined) who were genotyped for both the CFH rs1061170 variant and the LOC387715 rs10490924 variant in our data set. Regarding the CFH variant, 29.8% of the individuals possessed no variant alleles (TT genotype), 53.8% possessed one variant allele (TC genotype), and 16.4% possessed two variant alleles (CC genotype). Of the same 200 individuals with respect to the LOC387715 variant, 56.3% had no variant alleles (GG genotype), 35.6% had one variant allele (GT genotype), and 8.2% had both variant alleles (TT genotype). The breakdown into the five genotype combinations considered in this study was as follows: group 1, 34 individuals (17.0%); group 2, 27 individuals (13.5%); group 3, 74 individuals (37.0%); group 4, 62 individuals (31.0%); and group 5, 3 individuals (1.5%). Limited conclusions can be drawn from this sample size, but it does appear that a minority of individuals without AMD possess either or both of these risk variants.

Determination of high-risk genotypes and phenotypes could prove valuable to the clinician. If these associations are confirmed, rapid and cost-effective assays can be developed for determining whether an individual has the T allele at LOC387715 (rs10490924) and/or the C allele at CFH (rs1061170) and preventative and therapeutic treatments may be recommended based on an individual's phenotype as well as genotype. A more thorough understanding of the genotype-phenotype relationship in AMD may improve therapeutic recommendations, provide a more accurate diagnosis, make the investigation of the non-genetic components more straightforward, and allow for a better understanding of the mechanism of this complex disease.

### REFERENCES

The disclosure of each reference cited is expressly incorporated herein for the purpose to which is referenced in the text.
1. Klein R, Peto T, Bird A, Vannewkirk M R. The epidemiology of age-related macular degeneration. Am J. Ophthalmol. 2004; 137:486-495.
2. Age-Related Eye Disease Study Research Group. A randomized, placebo-controlled, clinical trial of high-dose supplementation with vitamins C and E, beta carotene, and zinc for age-related macular degeneration and vision loss: AREDS report No. 8. Arch Ophthalmol. 2001; 119:1417-1436.
3. Schmidt S, Scott W K, Postel E A, et al. Ordered subset linkage analysis supports a susceptibility locus for age-related macular degeneration on chromosome 16p12. BMC Genet. 2004; 5:18.
4. Magnusson K P, Duan S, Sigurdsson H, et al. CFH Y402H confers similar risk of soft drusen and both forms of advanced AMD. PLoS Med. 2006; 3:e5.
5. Haines J L, Hauser M A, Schmidt S, et al. Complement factor H variant increases the risk of age-related macular degeneration. Science. 2005; 308:419-421.
6. Klein R J, Zeiss C, Chew E Y, et al. Complement factor H polymorphism in age-related macular degeneration. Science. 2005; 308:385-389.
7. Edwards A O, Ritter R III, Abel K J, et al. Complement factor H polymorphism and age-related macular degeneration. Science. 2005; 308:421-424.
8. Postel E A, Agarwal A, Caldwell J, et al. Complement factor H increases risk for atrophic age-related macular degeneration. Ophthalmology. 2006; 113:1504-1507.
9. Shuler R K, Gallins P, Hauser M A, et al. Peripheral reticular pigmentary change is associated with complement factor H polymorphism (Y402H) in age-related macular degeneration. Paper presented at: 2006 Joint Meeting of the American Academy of Ophthalmology and the Asian Pacific Academy of Ophthalmology; Nov. 12, 2006; Las Vegas, Nev.
10. Rivera A, Fisher S A, Fritsche L G, et al. Hypothetical LOC387715 is a second major susceptibility gene for age-related macular degeneration, contributing independently of complement factor H to disease risk. Hum Mol Genet. 2005; 14:3227-3236.
11. Schmidt S, Hauser M A, Scott W K, et al. Cigarette smoking strongly modifies the association of LOC387715 and age-related macular degeneration. Am J Hum Genet. 2006; 78:852-864.
12. Postel E A, Agarwal A, Schmidt S, et al. Comparing age-related macular degeneration phenotype in probands from singleton and multiplex families. Am J. Ophthalmol. 2005; 139:820-825.
13. De la Paz M A, Pericak-Vance M A, Haines J L, Seddon J M. Phenotypic heterogeneity in families with age-related macular degeneration. Am J. Ophthalmol. 1997; 124:331-343.
14. Age-Related Eye Disease Study Research Group. The Age-Related Eye Disease Study system for classifying age-related macular degeneration from stereoscopic color fundus photographs: the Age-Related Eye Disease Study report number 6. Am J. Ophthalmol. 2001; 132:668-681.
15. Age-Related Eye Disease Study Research Group. The Age-Related Eye Disease Study (AREDS): design implications: AREDS report no. 1. Control Clin Trials. 1999; 20:573-600.
16. Chylack L T Jr, Wolfe J K, Singer D M, et al, Longitudinal Study of Cataract Study Group. The Lens Opacities Classification System III. Arch Ophthalmol. 1993; 111:831-836.

We claim:

1. A method to assess risk of neovascular AMD in a patient comprising:
assaying a sample of patient DNA or protein and determining whether the patient has a T allele at rs10490924; and
identifying a patient with the T allele as being at higher risk of neovascular AMD than geographical atrophy.

2. The method of claim 1 further comprising:
identifying a patient with two T alleles at rs 10490924 as having an earlier predicted onset of neovascular AMD than a patient with one T allele at rs10490924.

3. A method to assess risk of neovascular AMD in a patient comprising:
assaying a sample of patient DNA or protein and determining whether the patient has a T allele at rs10490924; and
identifying a patient with the T allele as being at higher risk of neovascular AMD than a patient without the T allele.

4. The method of claim 1 further comprising:
identifying a patient with two T alleles at rs 10490924 as having an earlier predicted onset of neovascular AMD than a patient with no T alleles at rs10490924.

5. The method of claim 1 or 3 further comprising the step of:
prescribing a medicament, supplement, or diet to the patient with a T allele at rs10490924 to slow progression or delay onset of neovascular AMD.

6. The method of claim 1, 2, 3, or 4 wherein the patient is not a cigarette smoker.

7. The method of claim 1, 2, 3, or 4 wherein the patient is a cigarette smoker.

8. The method of claim 1 or 3 wherein the T allele is determined by identifying an A69S protein.

9. The method of claim 8 wherein an antibody is used to identify an A69S protein.

10. The method of claim 9 wherein an enzyme-linked immunosorbent assay is used to identify an A69S protein.

11. The method of claim 1 or 3 wherein a variant coding sequence is determined.

12. The method of claim 11 wherein a polymerase chain reaction is used to amplify a region of said DNA of said patient containing the T allele at rs 10490924.

13. The method of claim 12 wherein a real-time polymerase chain reaction assay is used.

14. The method of claim 11 wherein primer mass extension and matrix-assisted laser desorption ionization-time-of-flight mass spectrometry analysis are used to determine a variant coding sequence.

15. The method of claim 11 wherein nucleic acid sequencing is used to determine a variant coding sequence.

16. The method of claim 11 wherein a molecule of DNA comprising nucleotide 26 and at least 15 additional contiguous nucleotides of SEQ ID NO: 1 is synthesized.

17. The method of claim 11 wherein a DNA molecule comprising the sequence shown in SEQ ID NO: 1 or its complement is degraded and its degradations products are analyzed.

18. A method to determine an appropriate regimen to prescribe to a patient for delaying onset of neovascular AMD in a patient comprising:
assaying a sample of patient DNA or protein and determining whether the patient has two T alleles at rs10490924 in LOC687715 using a real-time polymerase chain reaction assay;
identifying the patient as having an earlier predicted onset of neovascular AMD than a patient with one or no T allele at rs10490924 if the patient has two T alleles at rs10490924; and
prescribing a medicament, supplement, or diet to the patient with two T alleles at rs10490924 to delay onset or to slow progression of neovascular AMD.
